(19) United States
(12) Patent Application Publication
(10) Pub. No.: US 2009/0226542 A1
(43) Pub. Date: Sep. 10, 2009

(54) FITNESS AND EXERCISE-ENHANCING TABLET CONTAINING PROTEIN ISOLATE

(76) Inventor: Andrea K. Cresta, Candia, NH (US)

Correspondence Address: Russ Weinzimmer, 614 Nashua Street, Suite 53, Milford, NH, 03055, US

(21) Appl. No.: 12/467,260

(22) Filed: May 16, 2009

Related U.S. Application Data
(63) Continuation-in-part of application No. 12/253,992, filed on Oct. 19, 2008.
(60) Provisional application No. 61/017,576, filed on Dec. 29, 2007.

(51) Int. Cl. A61K 33/06; A61K 38/16; A61K 33/00

(52) U.S. Cl. 424/682; 514/12; 424/722

(57) ABSTRACT

An exercise-enhancing food tablet, preferably a hard or chewable candy or mint, is disclosed for enhancing exercise and physical fitness when ingested before, during, and/or after exercise. The edible tablet includes a mixture of carbohydrate and protein, the protein portion being between 20% and 40% of the mixture by weight, and preferably between 25% and 33% of the mixture by weight. The protein portion is preferably whey protein, but can be any combination of whey protein, soy protein, and pea protein. The carbohydrate portion of the mixture is preferably 85% dextrose and 15% fructose, but can be any combination of dextrose, maltodextrin, fructose, and sucrose. In preferred embodiments, sodium, potassium, magnesium, vitamin C, and/or caffeine are also included. The tablets can also include a flavoring, such as spearmint, peppermint, or fruit flavoring. In preferred embodiments the tablets weigh substantially 2 grams, and an adult serving is eight tablets.

### CROSS-REFERENCES TO RELATED APPLICATIONS

This application is a Continuation-In-Part of U.S. patent application Ser. No. 12/253,992 entitled “FITNESS AND EXERCISE-ENHANCING TABLET CONTAINING PROTEIN ISOLATE,” filed Oct. 19, 2008, herein incorporated by reference.

U.S. patent application Ser. No. 12/253,992 is entitled to the benefit of U.S. Provisional Patent Application Number 61/017,576 entitled “EXERCISE-ENHANCING MINT/HARD CANDY CONTAINING PROTEIN”, filed Dec. 29, 2007, herein incorporated by reference.

### FIELD OF THE INVENTION

This invention relates to health foods, and more particularly to fitness and exercise-enhancing foods in tablet form.

### BACKGROUND

Exercise can be enjoyable, and provides many well known benefits for the health and well being of exercisers. However, exercise places significant demands on the body by consuming energy and stressing muscles. Proper nutritional support can be of significant help in prolonging exercise and increasing its benefits and enjoyment by providing carbohydrates for energy and/or protein for the support and repair of muscles.

There are many different kinds of health food available for the benefit of health and fitness conscious people. In particular, various convenience health and fitness foods, such as nutrition bars and energy drinks, are available for the convenience of exercisers who require extra energy and/or protein before, during, or immediately after exercise.

However, some of these convenience health and fitness foods are bulky and difficult to carry. They are also difficult to eat in the midst of continuous activity, thereby requiring that an exerciser pause, at least briefly, so as to consume the exercise food. These are significant limitations for exercisers who do not wish to carry bulky items, or to interrupt their exercise routines or their post-exercise activities, even for the time it may take to eat a nutrition bar or drink a so-called “energy drink” or “sports drink.” Nutritional drinks in particular can also be upsetting to the stomach if consumed during or immediately before or after vigorous exercise, especially if consumed suddenly and quickly.

Various exercise supporting tablets have been introduced in the prior art which are easy to carry and easy to ingest before, during, and/or after exercise. Some of these tablets contain valuable electrolytes and vitamins which can boost an exerciser's energy levels and enhance the exerciser's performance during exercise. Some tablets further include carbohydrates, and/or sweeteners for energy enhancement and/or enhanced taste. However, these prior art tablets do not provide ingredients that enhance muscle support and repair.

### SUMMARY OF THE INVENTION

An edible health and fitness food in the form of a tablet such as a candy, mint, capsule, or pill containing both carbohydrates and protein is claimed for supporting and enhancing exercise and physical fitness. The fitness food of the present invention can be consumed before, during, and/or after exercise, and can be in the form of a “hard” item that is dissolved in the mouth, a chewable item, or a tablet that is swallowed whole.

The tablet of the present invention includes a mixture of carbohydrate and protein, wherein the protein portion is between 20% and 40% of the mixture by weight, and preferably between 25% and 33% of the mixture by weight. The protein portion of the mixture can be any combination of whey protein, soy protein, and pea protein, and the carbohydrate portion of the mixture can be any combination of dextrose, maltodextrin, fructose, and sucrose. In preferred embodiments, sodium, potassium, magnesium, vitamin C, and/or caffeine are also included in the tablet.

The claimed exercise-enhancing tablet offers advantages over the prior art by including not only carbohydrates but also intact protein, which in preferred embodiments includes at least one of whey protein isolate, soy protein isolate, and pea protein isolate. The intact protein metabolizes more efficiently than free amino acids, reducing the breakdown of muscle and keeping muscle tissues stronger during exercise. The exercise-enhancing tablet preferably contains a blend of carbohydrates, as well as other vitamins, minerals and electrolytes, all of which provide nutrients necessary for optimal fitness and exercise performance.

In preferred embodiments, a unique blend of carbohydrates acts to reduce the amount of simple sugars, and also increases the amount of carbohydrate uptake in the body. Dextrose, and in some embodiments maltodextrin, break down in the intestines and are efficiently transported into the circulatory system as glucose, thereby enhancing performance during aerobic exercise. Fructose is transported by its own transporter, increasing the rate of carbohydrate uptake and reducing the body's reliance on other fuel sources during exercise, thereby postponing exhaustion and fatigue and extending exercise time. In some preferred embodiments, the claimed exercise-enhancing tablet includes a flavoring, so as to make the tablet more palatable.

### DETAILED DESCRIPTION OF PREFERRED EMBODIMENTS

The present invention is an exercise-enhancing tablet that can be dissolved in the mouth, chewed, or swallowed, so as to provide nutritional support to an exerciser before, during, and/or after exercise. The tablet can be in the form of a typical, medicinal “pill” or capsule, but is preferably in the form of a hard candy or chewable mint.

The exercise-enhancing tablet includes a mixture of carbohydrate and protein, wherein the protein portion is between 20% and 40% of the mixture by weight, and preferably is between 25% and 33% of the mixture by weight. The protein portion of the mixture can be any combination of whey protein, soy protein, and pea protein, and the carbohydrate portion of the mixture can be any combination of dextrose, maltodextrin, fructose, and sucrose. Nutritional support is provided by including at least one carbohydrate as well as a complete protein, thereby supplying energy for use during exercise and protein for support and repair of muscles. In preferred embodiments, sodium, potassium, magnesium, vitamin C, and/or caffeine are also included in the tablet.

For a tablet weighing substantially 2 grams, and having an adult serving size of 8 tablets, the formulation of a preferred embodiment is presented in Table 1, Table 2, and Table 3 below, and a best-mode formulation is presented in Table 4 below:

TABLE 1
Ingredients Included in a Preferred Embodiment

| Ingredient | Quantity Range | Units |
|---|---|---|
| Dextrose | 0.9-1.2 | grams |
| Fructose | 0.10-0.14 | grams |
| Whey Protein | 0.4-0.6 | grams |

TABLE 2
Electrolytes and Vitamins Included in a Preferred Embodiment
(in addition to carbohydrates and protein)

| Ingredient | Quantity Range | Units |
|---|---|---|
| Sodium | 0-120 | milligrams |
| Magnesium | 0-65 | milligrams |
| Potassium | 0-65 | milligrams |
| Vitamin C | 0-50 | milligrams |

TABLE 3
Caffeine Included in a Preferred Embodiment (in addition
to carbohydrates, protein, electrolytes and vitamins)

| Ingredient | Quantity Range | Units |
|---|---|---|
| Caffeine | 0-15 | milligrams |

TABLE 4
Ingredients Included in a Best Mode Embodiment

| Ingredient | Quantity Range | Units |
|---|---|---|
| Dextrose | 1.17 | grams |
| Fructose | 0.129 | grams |
| Whey Protein | 0.5 | grams |
| Magnesium | 0.705 | milligrams |
| Potassium | 7.24 | milligrams |
| Calcium | 0.639 | milligrams |

In preferred embodiments such as the ones presented in Table 1 and Table 4, whey protein isolate is included in the tablets, since whey protein isolate has been shown to be a rapidly metabolized protein source, thereby making it ideal for providing muscle tissue with amino acids in a timely manner. It can be seen from Table 2 that electrolytes and Vitamin C are included in the formulation of some preferred embodiments, so as to further boost an exerciser's energy levels and enhance the exerciser's performance during exercise. It can be seen from Table 3 that caffeine is also added in the formulation of some preferred embodiments, to further increase energy levels and enhance performance during exercise.

Similar preferred embodiments include flavoring, such as fruit flavor, peppermint, or spearmint, so as to make the mints more enjoyable to consume. In fact, some embodiments include sufficient flavoring to be eaten purely for enjoyment.

Other modifications and implementations will occur to those skilled in the art without departing from the spirit and the scope of the invention as claimed. Accordingly, the above description is not intended to limit the invention except as indicated in the following claims.

What is claimed is:

1. An exercise-enhancing tablet comprising:
a mixture of carbohydrate and protein:
between 20% and 40% of the mixture by weight being protein;
the carbohydrate including at least one of dextrose, maltodextrin, sucrose, and fructose; and
the protein includes at least one of soy protein, pea protein, and whey protein.

2. The exercise-enhancing tablet of claim 1, further comprising at least one of:
up to 8% by weight of sodium;
up to 5% by weight of magnesium;
up to 5% by weight of potassium; and
up to 3% by weight of vitamin C.

3. The exercise-enhancing tablet of claim 1, further comprising from 3% to 10% by weight of caffeine.

4. The exercise-enhancing tablet of claim 1, wherein the tablet is one of a hard candy and a mint.

5. The exercise-enhancing tablet of claim 1, wherein the tablet includes at least one flavoring.

6. The exercise-enhancing table of claim 5, wherein the flavoring tastes like one of mint and fruit.

7. The exercise-enhancing tablet of claim 5, wherein the flavoring tastes like one of spearmint and peppermint.

8. The exercise-enhancing tablet of claim 1, wherein the tablet is one of:
a hard tablet edible by dissolving in a mouth;
a soft tablet edible by chewing; and
a tablet edible by swallowing the tablet whole.

9. The exercise-enhancing tablet of claim 1, wherein the tablet has a weight of substantially 2 grams, and an adult serving size is eight tablets.

10. An exercise-enhancing tablet comprising:
a mixture of carbohydrate and whey protein, wherein:
between 25% and 33% of the mixture by weight is whey protein; and
the carbohydrate is composed of dextrose and fructose included by weight in a ratio of between 7:1 and 12:1 respectively.

11. The exercise-enhancing tablet of claim 10, further comprising at least one of:
up to 8% by weight of sodium;
up to 5% by weight of magnesium;
up to 5% by weight of potassium; and
up to 3% by weight of vitamin C.

12. The exercise-enhancing tablet of claim 10, further comprising from 3% to 10% by weight of caffeine.

13. The exercise-enhancing tablet of claim 10, wherein the tablet is one of a hard candy and a mint.

14. The exercise-enhancing tablet of claim 10, wherein the tablet includes at least one flavoring.

15. The exercise-enhancing table of claim 14, wherein the flavoring tastes like one of mint and fruit.

16. The exercise-enhancing tablet of claim 14, wherein the flavoring tastes like one of spearmint and peppermint.

17. The exercise-enhancing tablet of claim 10, wherein the tablet is one of:
a hard tablet edible by dissolving in a mouth;
a soft tablet edible by chewing; and
a tablet edible by swallowing the tablet whole.

18. The exercise-enhancing tablet of claim 10, wherein the tablet has a weight of substantially 2 grams, and an adult serving size is eight tablets.

19. An exercise-enhancing nutritional tablet comprising one-eighth of an adult serving, the tablet comprising substantially by weight:
1.17 grams of dextrose;
0.129 grams of fructose;
0.5 grams of whey protein;
0.705 mg of magnesium;
7.24 mg of potassium; and
0.639 mg of calcium.
